(19) United States
(12) Patent Application Publication
(10) Pub. No.: US 2011/0097263 A1
(43) Pub. Date: Apr. 28, 2011

(54) KIT FORMULATION FOR THE PREPARATION OF IMMUNOLIPOSOME DRUG IN COMBINED BIMODALITY RADIOCHEMOTHERAPY

(75) Inventors: WEI-CHUAN HSU, TAOYUAN COUNTY (TW); TE-WEI LEE, TAOYUAN COUNTY (TW); CHIA-YU YU, TAOYUAN COUNTY (TW); CHIH-HSIEN CHANG, TAOYUAN COUNTY (TW); TSUI-JUNG CHANG, TAOYUAN COUNTY (TW)

(73) Assignee: INSTITUTE OF NUCLEAR ENERGY RESEARCH ATOMIC ENERGY COUNCIL, EXECUTIVE YUAN, TAOYUAN COUNTY (TW)

(21) Appl. No.: 12/606,529

(22) Filed: Oct. 27, 2009

(51) Int. Cl. A61K 51/04; A61K 51/00

(52) U.S. Cl. 424/1.65; 424/1.11

(57) ABSTRACT

A kit formulation for the preparation of immunoliposome drug in combined chemotherapy and radionuclide therapy is disclosed, which consists: (1) a vial A containing proteins; (2) a vial B containing Traut's reagent; (3) a vial C containing DSPC, Cholesterol, mPEG-DSPE, Mal-DSPE-PEG and chemotherapy drug; (4) a vial D containing BMEDA, gluconate acetate, SnCl₂; (5) a vial E which containing radionuclide solution. The procedure using the kit comprises: (i) withdraw the radionuclide solution from the vial E; (ii) inject the solution into the vial D for enabling reaction; (iii) withdraw the contents of the vial B and inject into the vial A; (iv) withdraw the contents of the protein and Traut's reagent mixtures from the vial A and inject into the vial C for enabling reaction; (v) withdraw the contents of the immunoliposome from the vial C; (vi) inject the immunoliposome into the vial D of step (ii) for enabling reaction.

### FIELD OF THE INVENTION

This invention relates to manufacture a kit for the preparation of active targeting immunoliposome drug and the kit formulation is applied to combine bimodality radiochemotherapy for tumor and ascites.

### BACKGROUND OF THE INVENTION

Liposomal formulations serve as one of the promising approaches since the association of drugs with lipid carriers result in a dramatic improvement of the pharmacokinetics of the drug, resulting in reduced toxicities and improved therapeutic efficacies. Although rapid clearance of the conventional liposomes by the reticular endothelial system (RES) is recognized as one of the major drawbacks in anticancer drug delivery, this can be overcome by utilizing sterically stabilized liposomes. The surface of the liposomes can be modified with flexible hydrophilic polymers such as polyethylene glycol (PEG) which have been reported to spontaneously accumulate in solid tumors via the enhanced permeability and retention (EPR) effect through the passive targeting mechanism. Another approach for overcoming the limitations of the conventional liposomal formulations is to develop immunoliposomes which can actively target solid tumors by attaching monoclonal or polyclonal antibodies on the liposomal surface. Such formulations not only have the potential to transfer large number of drug molecules to an individual target site but also exerts similar or greater antitumor activities compared to the native drug.

Two diagnostic and therapeutic radionuclides, ¹⁸⁸Re and ¹⁸⁶Re, have excellent physical properties. They emit γ-ray and β particle and can use as diagnostic and therapeutic radionuclides. The energy of the γ-ray of ¹⁸⁸Re and ¹⁸⁶Re are 155 and 139 KeV, respectively. ^{99m}Tc is also an ideal radionuclide for diagnostic imaging because of it emits γ-ray (141 keV) only. The physical characteristics of ¹⁸⁸Re, ¹⁸⁶Re and ^{99m}Tc are shown in Table 1. Bao et al. have developed a direct labeling method using ^{99m}Tc-BMEDA complex to label the commercially available pegylated liposome doxorubicin. (J. Pharmacol Exp Ther, 308: 419-425, 2004). The product of kit for the preparation of ¹⁸⁸Re-(or ¹⁸⁶Re) BMEDA/DXR-Liposome and application in the treatment of tumor and ascites has not found yet. On the other hand, There are some studies of immunoliposomes carrying chemotherapy drugs have been reported, but no study with the targeted therapy using immunoliposomal radiopharmaceuticals and chemotherapy drugs. This invention demonstrates a new kit formulation for the preparation of immunoliposome drug in combined bimodality radiochemotherapy.

TABLE 1
Physical Characteristics of ¹⁸⁸Re and ¹⁸⁶Re Radionuclides.

| | | | | | β-ray | | | |
|---|---|---|---|---|---|---|---|---|
| | Physical | Mode | γ-ray | | Energy | | Range in | |
| | half-life | of | Energy | Abundance | (MeV) | | tissue (mm) | |
| Radionuclide | (T_{1/2}) | decay | (MeV) | (%) | Max. | Ave. | Max. | Ave. |
| ¹⁸⁸Re | 16.98 h | β⁻ (100) | 0.155 | 14.9 | 2.12 | 0.765 | 11 | 3.5 |
| ¹⁸⁶Re | 3.8 d | β⁻ (92) | 0.139 | 9 | 1.075 | 0.323 | 3.6 | 1.8 |
| | | EC (8) | | | | | | |
| ^{99m}Tc | 6.022 h | γ | 0.141 | 98.6 | | | | |

### SUMMARY OF THE INVENTION

The kit consists of five components: (1) The vial A which contains proteins. (2) The vial B which contains Traut's reagent. (3) The vial C which contains DSPC, Cholesterol, mPEG-DSPE, Mal-DSPE-PEG and chemotherapy drug. (4) The vial D which contains BMEDA, gluconate acetate, SnCl₂. (5) The vial E which contains radionuclide solution. The procedure of using the kit is as the follows: (i) Withdraw the contents of the radionuclide solution from the vial E. (ii) Inject the solution into the vial D, and the mixtures react in appropriate temperature. (iii) Withdraw the contents of the vial B and inject into the vial A. (iv) Withdraw the contents of the protein and Traut's reagent mixtures from the vial A and inject into the vial C, the mixtures react in appropriate temperature. (v) Withdraw the contents of the immunoliposome from the vial C. (vi) Inject the immunoliposome into the vial D of step (ii), and the mixtures react in appropriate temperature. The reconstituted solution in the vial D is applied to combine bimodality radiochemotherapy for tumor and ascites.

The product of kit in this invention for preparation of immunoliposome drugs in combined bimodality radiochemotherapy has proved to be more simple, convenient, effective and easier than the prior art is.

### BRIEF DESCRIPTION OF THE DRAWINGS

FIG. 1A˜FIG. 1D is Cellular uptake of bimodality immunoliposome.

FIG. 2A˜FIG. 2B is Fluorescence confocal microscopy of bimodality immunoliposome.

FIG. 3 is Cellular retention of DXR-IL-C225 in A431 cell line.

FIG. 4. is Cellular retention of ¹⁸⁸Re-IL-C225 in A431 cell line.

FIG. 5. is Cytotocixity assay of ¹⁸⁸Re-DXR-IL-C225 in A431 cell line.

### DESCRIPTION OF THE INVENTION

The following abbreviations are employed:
Mal-DSPE-PEG: N-[(3-Maleimido-1-oxopropyl) aminopropyl polyethyleneglycol-carbamyl]distearoylphosphatidyl-ethanolamine
BMEDA: N,N-bis(2-mercaptoethyl)-N′,N′-diethylethylenediamine
DSPC: Distearoyl phosphatidylcholine
PEG: Polyethylene glycol
DSPE: Distearyl phosphatidylethanolamine
Traut's: 2-Iminothiolane-HCl
IL: Immunoliposome

### Example 1

### The Preparation and Quality Control of Vial D and E Components

Five mg of BMEDA and 0.5 mL of 0.17 mol/L glucohepatonate dissolved in 10% acetate solution were added into vial D. Then, flushing with N₂ gas for 1 mins, followed by the addition of 120 μL (10 μg/μL) of stannous chloride. The vial E (for example, ¹⁸⁸Re solution) was added to the vial D (BMEDA, SnCl₂, Gluconate-acetate) and incubated at 80° C. for 1 h in water bath. The labeling efficiency could reach 99±1.73% (Rf: 1, free ¹⁸⁸Re; Rf: 0, ¹⁸⁸Re-BMEDA) by radio-TLC.

### Example 2

### The Preparation and Quality Control of Vial C Component

DSPC, cholesterol, PEG₂₀₀₀-DSPE and DSPE-PEG-Maleimide (molar ratio 3:2:0.3:0.24) were dissolved in 8 mL chloroform and placed in a 250 mL round-bottomed flask. The solvent was removed by rotary evaporation under reduced pressure at 60° C. Then the resulting dried thin film was hydrated in a 5 mL 250 mM ammoniumsulfate solution (250 mM (NH₄)₂SO₄, pH 5.0, 530 mOsm) and dispersed by hand shaking at 60° C. The resulting suspension of multilamellar vesicles was then frozen and thawed 6 times, followed by repeated extrusion through polycarbonate membrane filters using high-pressure extrusion equipment (Lipex Biomembrane, Vancouver, Canada) at 60° C. The extra-liposomeal salt was removed by gel filtration on Sephadex G-50 column.

Doxorubicin stock (10 mg/mL dissolved in ddH₂O) was add immediately into the solution as soon as liposome were eluted from gel foltration column described above at a concentration of 140 μg doxorubicin per μmole phospholipid. The mixture of liposome and doxorubicin was incubated in a 60° C. water bath for 30 mins agitation (100 rpm). After loading, unencapsulated doxorubicin was removed by Sephadex G-50 gel filtration column equilibrated with 0.9% NaCl solution. The eluted liposome solution was concentrated by ultracentrifugation at 150000×g for 90 mins. Then resuspend liposome precipitate with 0.9% NaCl solution. Liposomes were sterilized by filtration through 10.22 mm sterile filter and filled into vial B. The vial B component of quality control was as follows:
1. Vesicles were measured by dynamic laser scattering with a submicron particles analyzer (model nano-ZX, Malvern). Particle sizes ranged from 75-95 nm in diameter.
2. The amount of doxorubicin trapped inside the liposome was determine with a spectrofluorometer (FP6200, JASCO) at an excitaion wavelength of 475 nm and an emission wavelength of 580 nm. Doxorubicin loaded liposomes contained 2 mg doxorubicin per liposome solution.

### Example 3

### The Preparation and Quality Control of Vial A and B Component

2.5 mg of protein or antibody dissolve in degassed HEPES buffer (20 mM HEPES, 140 mM NaCl, 2 mM EDTA, pH 8.0) (vial A) were thiolated for 1 h at room temperature by reacting with 5-fold excess of Traut's reagent (2-Iminothiolane-HCl) (vial B) in degassed HEPES buffer.

### Example 4

### The Preparation and Quality Control of Chemotherapy Drugs Loaded Immunoliposome

For direct coupling of protein to liposomes, the thiolated protein (vial A products describe in Example 3) was added to preformed liposomes (vial C) at 40 μg protein per micromole of lipids, and then gently shaken at room temperature for 4 h under N₂. The final products (immunoliposome-doxorubicin, IL-DXR) were performed to determine the coupling efficiency by Bradford protein assay.

### Example 5

### The Preparation and Quality Control of Bimodality Immunoliposome

The IL-DXR (vial C products describe in Example 3) were added to the ¹⁸⁸Re-BMEDA (50-250 MBq) (vial D products describe in Example 1) solution and incubation at 60° C. for 30 min. The ¹⁸⁸Re-DXR-immunoliposome (¹⁸⁸Re-DXR-IL) (from D vial) solution was separated from free ¹⁸⁸Re-BMEDA using PD-10 column eluted with normal saline. Each 0.5 ml fraction was collected into a tube. The red color of ¹⁸⁸Re-DXR-IL was used to visually monitor the collection of the ¹⁸⁸Re-BMEDA/DXR-IL. The encapsulating efficiency was determined by using the activity in ¹⁸⁸Re after separation divided by the total activity before separation. The encapsulating efficiency was 40-60%.

### Example 6

### Cellular Uptake of Bimodality Immunoliposome

In this example, we use ¹⁸⁸Re-DXR-IL-C225 as an example to show the cellular uptake of bimodality immunoliposome. C225 (Erbitux®, CetuxiMAb) is a targeted therapy that targets and binds to the epidermal growth factor receptors (EGFR) on the surface of the cell. EGFR is found on the surface of many normal and cancer cells. By binding to these receptors, C225 blocks an important pathway that promotes cell division this result in inhibition of cell growth and apoptosis (cell suicide). C225 is used to treat metastatic colorectal cancer (cancer spread beyond the colon or rectum) that over-expresses the epidermal growth factor receptor (EGFR). It also approved for the treatment of squamous cell carcinoma of the head and neck. Cellular uptake experiments were performed at both 37° C. and 4° C. to study the effect of the receptor-mediated endocytosis exerted by C225-conjugated immunoliposome. Briefly, A431 human epidermoid carcinoma (EGFR overexpressed) and COLO 205 human colorectal adenocarcinoma cells (EGFR low expressed) were suspended in each culture medium at a density of 1×10⁶ cells/tube in 1.5 ml eppendorf. After 24 h, medium was changed with 1 mL of each culture media containing 1.5 μCi of ¹⁸⁸Re-DXR-IL-C225, ¹⁸⁸Re-DXR-IL-IgG (for non-specific control) or ¹⁸⁸Re-DXR-liposomes (¹⁸⁸Re-DXR-Ls). Each cell in triplicate was incubated for 2 h at either 37° C. or 4° C. For the blocking study, 100 Ξg/tube of C225 was added to tube simultaneously. The cells were washed with cold PBS twice to remove unbound immunoliposome. After centrifugation at 13,000 rpm for 5 min, the supernatant removed carefully. Cellular uptake efficiency was calculated using the following formula:

$$= \frac{Activity of {}^{188} Re in the cells pellet after 2 h incubation}{Total activity of {}^{188} Re added to the cells} \times 100 \%$$

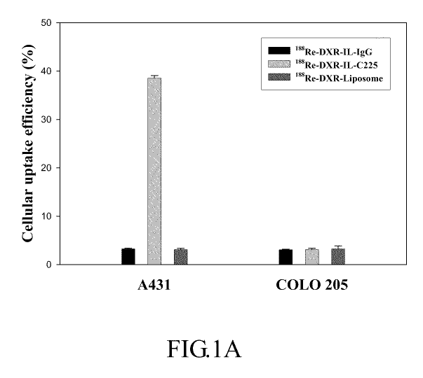

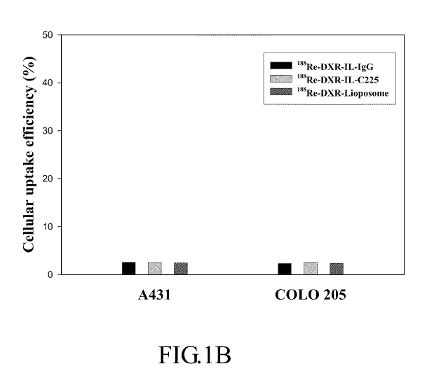

¹⁸⁸Re-DXR-IL-C225 showed higher cellular uptake (about 20 times) in EGFR over-expressing cancer cells (A431) at 37° C. than that in the ¹⁸⁸Re-DXR-IL-IgG or the ¹⁸⁸Re-DXR-Ls (FIG. 1A and FIG. 1B). However, in EGFR low-expressing cells (COLO 205), a significant difference was not observed. Cellular uptake efficiency is similar in three drugs in COLO 205 cell line (about 2%). Moreover, the specific binding of ¹⁸⁸Re-DXR-IL-C225 was observed only in A431 at 4° C. (Cellular uptake efficiency is about 20%) (FIG. 1C and FIG. 1D). The higher cellular uptake of ¹⁸⁸Re-DXR-IL-C225 can be block by excess cold C225 (FIG. 1B and FIG. 1D). These results suggest that the cellular uptake of ¹⁸⁸Re-DXR-IL-C225 is related to the receptor-mediated endocytosis mechanism.

### Example 7

### Fluorescence Confocal Microscopy of Bimodality Immunoliposome

For fluorescence microscopy, DXR-IL-C225 liposomes were prepared as described above using Vial A, B and C. Cells grown on two chamber coverslips were incubated at 37° C. or 4° C. with DXR-IL-C225, DXR-IL-IgG or DXR-Ls. After incubation, The cells fixed with 2% formaldehyde, mounted in glycerol, and observed with OLYPUS FLUOVIEW FV300 confocal microscope.

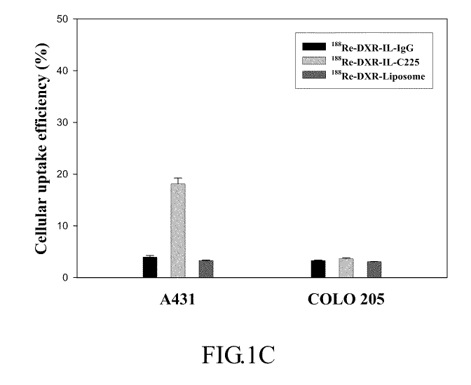

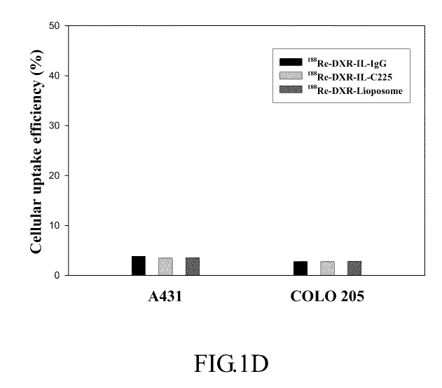

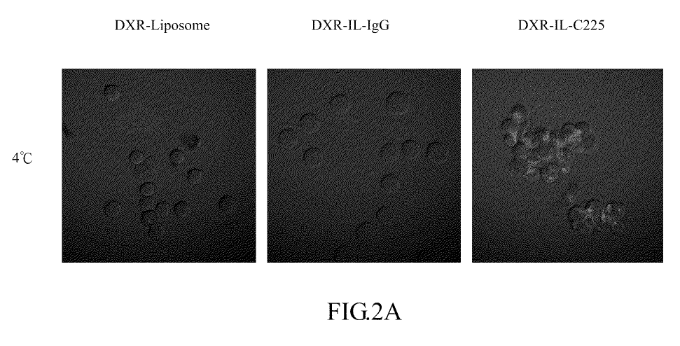

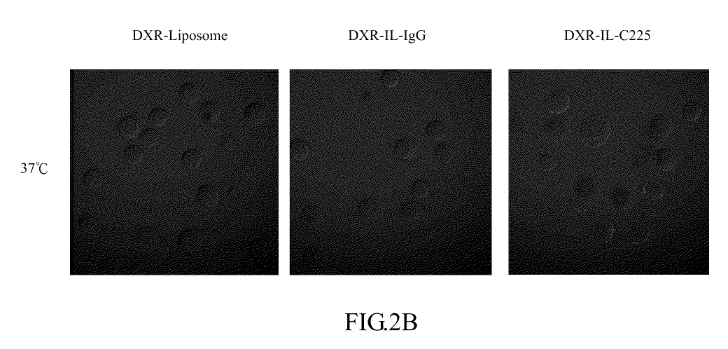

Cellular association and localization of DXR-labeled liposomes in A431 cell lines were observed by confocal laser scanning microscopy. The DXR-IL-C225 quickly attached on the surface of the EGFR receptor over-expressing cell lines, and then entered into the cells after 1 h. However, the incubation of DXR-IL-IgG or DXR-Ls with A431 did not show any detectable fluorescence (FIG. 2A), which implies that the DXR-IL-C225 binds specifically to the EGFR receptor and is efficiently internalized by the EGFR receptor-mediated endocytosis. Furthermore, DXR-IL-C225 show the specific binding with the cell surface clearly when the cells incubated at 4° C. (FIG. 2B).

### Example 8

### Cellular Retention of ¹⁸⁸Re-DXR-IL-C225 in A431 Cell Line

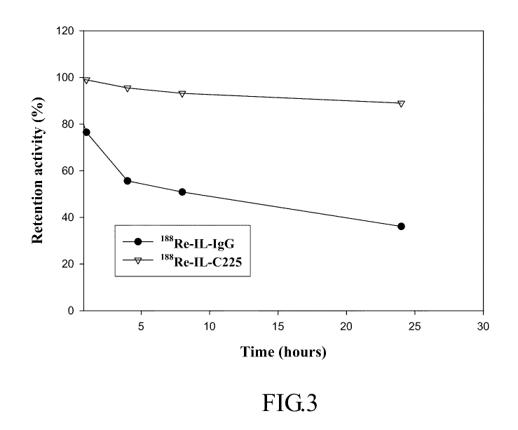

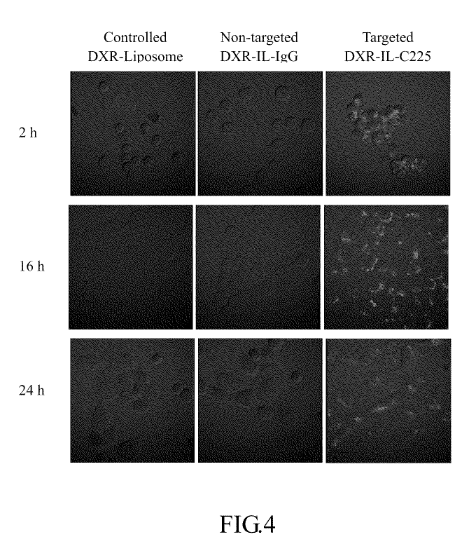

In this example, we use ¹⁸⁸Re-IL-C225 as an example to show the Cellular retention of chemotherapy drug or radionuclide in the bimodality immunoliposome. For radionuclide drug retention, approximately 1×10⁶ A431 cells were resuspended in 1.5 ml eppendorf. ¹⁸⁸Re-IL-C225 and/or ¹⁸⁸Re-IL-IgG (22 ng/ml, 1.5 nM), were diluted in DMEM medium with 10% FBS and added to prewashed cells with a total volume of 0.5 ml/tube. ¹⁸⁸Re-IL-C225 and/or ¹⁸⁸Re-IL-IgG were incubated at 37° C. for 2 h. All cells were then washed six times in cold PBS and further incubated with 1 ml fresh medium for 0, 1, 2, 4, 8 and 24 h. After incubation, the medium was collected, the cells were washed and counted and the radioactivity was measured as described earlier. The cellular retention of ¹⁸⁸Re after delivery as ¹⁸⁸Re-IL-C225 and/or ¹⁸⁸Re-IL-IgG was studied at various times. The remaining cell cell-associated radioactivity after 24 h of incubation was 36% for ¹⁸⁸Re-IL-IgG and 89% for ¹⁸⁸Re-IL-C225 (FIG. 3). For chemotherapy drug retention, the similar procedures were performed. Approximately 1×10⁶ A431 cells were resuspended in 1.5 ml eppendorf. DXR-IL-C225, DXR-IL-IgG and DXR-Ls (5 mM of phospholipids concentration) were incubated at 37° C. for 2 h. After incubation, all cells were then washed six times in cold PBS and further incubated with 1 ml fresh medium for 2, 16 and 24 h. After incubation, the cells fixed with 2% formaldehyde, mounted in glycerol, and observed with OLYPUS FLUOVIEW FV300 confocal microscope. The remaining cell cell-associated chemotherapy drug after 24 h of incubation was demonstrated clearly. (FIG. 4).

### Example 9

### Cytotocixity Assay of ¹⁸⁸Re-DXR-IL-C225 in A431 Cell Line

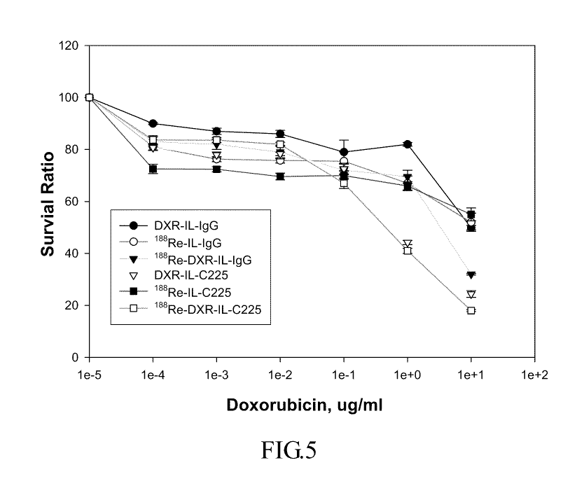

The cytotoxic activity of ¹⁸⁸Re-DXR-IL-C225 on A431 cells was measured with an WST-1 (4-[3-(4-Iodophenyl)-2-(4-nitrophenyl)-2H-5-tetrazolio]-1,3-benzene disulfonate) assay kit (Roche Diagnostics, Mannheim, Germany). Adherent A431 cells (3×10³/well) were treated with a medium containing ¹⁸⁸Re-DXR-IL-C225, ¹⁸⁸Re-IL-C225, DXR-IL-C225, ¹⁸⁸Re-DXR-IL-IgG, ¹⁸⁸Re-IL-IgG and DXR-IL-IgG at 37° C. for 1 h. The concentration of DXR is range from 1×10⁵-10 μg/ml. The concentration of DXR is 10 μCi//ml. After washing, the treated cells were further incubated for 3 days and 10% of WST-1 reagent was added for 1 h before assay. The formazan formation was extracted quantified in an ELISA Reader at 450 nm. ¹⁸⁸Re-DXR-IL-C225 shows a 84% decrease of cell viability in EGFR overexpressing cancer cells (FIG. 5). This example demonstrated that nano-targeted bimodality immunoliposome (¹⁸⁸Re-DXR-IL-C225) is superior to other formulations (¹⁸⁸Re-IL-C225, DXR-IL-C225, ¹⁸⁸Re-DXR-IL-IgG, ¹⁸⁸Re-IL-IgG and DXR-IL-IgG) and is potentially to treat with patients with cancers.

We claim:

1. A Kit formulation, comprising:
(1) Vial A which contains proteins; (2) The vial B which contains Traut's reagent; (3) The vial C which contains DSPC, Cholesterol, mPEG-DSPE, Mal-DSPE-PEG and chemotherapy drug; (4) The vial D which contains BMEDA, gluconate acetate, SnCl₂; (5) The vial E which contains radionuclide solution.

2. The kit composition according to claim 1, wherein said protein is monoclonal antibody or peptide, anti-EGFR monoclonal antibody, anti-VEGF monoclonal antibody, anti-PDGF monoclonal antibody.

3. The kit composition according to claim 1, wherein said Traut's reagent is 2-Iminothiolane-HCl.

4. The kit composition according to claim 1, wherein said chemotherapy drug is doxorubicin, daunorubicin, vinolbine, palitaxol, fluorouracil, As₂O₃.

5. The kit composition according to claim 1, wherein said radionuclide solution is ^{99m}Tc, ¹⁸⁶Re or ¹⁸⁸Re.

6. The process for using the kit, comprising the step of (i) Withdraw the contents of the radionuclide solution from the vial E; (ii) Inject the solution into the vial D, and the mixtures react in appropriate temperature; (iii) Withdraw the contents of the vial B and inject into the vial A; (iv) Withdraw the contents of the protein and Traut's reagent mixtures from the vial A and inject into the vial C, the mixtures react in appropriate temperature. (v) Withdraw the contents of the immunoliposome from the vial C. (vi) Inject the immunoliposome into the radionuclide labeled BMEDA of the vial D of step (ii), and the mixtures react in appropriate temperature; and (vii)
The reconstituted solution is obtained in the vial D.

7. The process according to claim 6, wherein said radionuclide solution is ^{99m}Tc, ¹⁸⁶Re or ¹⁸⁸Re.

8. The process according to claim 6, wherein said the radionuclide labeled BMEDA is ^{99m}Tc-BMEDA, ¹⁸⁶Re-BMEDA or ¹⁸⁸Re-BMEDA.

9. The process according to claim 6, wherein said appropriate temperature is 20-100° C.

10. The process according to claim 6, wherein said the reconstituted solution is ^{99m}Tc-BMEDA/CHEM-Immunoliposome, ¹⁸⁶Re-BMEDA/CHEM-Immunoliposome or ¹⁸⁸Re-BMEDA/CHEM-Immunoliposome.

11. The process according to claim 10, wherein said CHEM presents chemotherapy drug which is doxorubicin, daunorubicin, vinolbine, palitaxol, fluorouracil, or As₂O₃.

12. The kit is applied to combine active targeting radionuclide therapy and chemotherapy for imaging and treatment of tumor and ascites.
